(19) United States
(12) Patent Application Publication
(10) Pub. No.: US 2025/0040975 A1
(43) Pub. Date: Feb. 6, 2025

(54) HOLDERS, KITS, AND METHODS

(71) Applicant: GLW, Inc., Englewood Cliffs, NJ (US)

(72) Inventors: Axel Cremer, Fahrenkrug (DE); Garret Mauldin, Erie, CO (US); Yash Dalal, Englewood Cliffs, NJ (US)

(73) Assignee: GLW, Inc., Englewood Cliffs, NJ (US)

(21) Appl. No.: 18/794,430

(22) Filed: Aug. 5, 2024

Related U.S. Application Data
(60) Provisional application No. 63/530,945, filed on Aug. 4, 2023.

(51) Int. Cl. A61B 17/86; B65D 81/02

(52) U.S. Cl. CPC A61B 17/865; B65D 81/025

(57) ABSTRACT

Holders for bone screws, kits including a holder and one or more bone screw, and methods of loading a screw onto a driver for driving the screw into a bone are described. A holder for a bone screw includes a main body having a first surface and a second surface. The main body defines a retainer having first and second arms, each of which is a flap of the main body that is movable from a first configuration, in which the respective arm is coplanar with the adjacent portions of the main body, and a second configuration in which the respective arm extends away from the adjacent portions of the main body. The first and second arms are biased toward returning to the first configuration in which the respective arm is coplanar with adjacent portions of the main body.

### FIELD

The disclosure relates to the field of holders for objects to be retained, such as screws and other fasteners, and related kits and methods. More particularly, the disclosure relates to holders for implantable medical fasteners, such as bone screws, kits including a holder and one or more medical fasteners, and methods of loading a medical fastener onto a tool for implanting the medical fastener into a patient. Selected example embodiments are described and relate to bone screw holders having one or more collapsible and biased retainers for holding one or more bone screws and for providing a resistive force on a portion of the one or more bone screws during loading onto a driver.

### BACKGROUND

One challenge in working with objects that require another tool for positioning, such as fasteners that require a driver or other tool for final positioning of the fasteners, is the need to engage the object on the tool. Direct manipulation of the object by hand to engage the object with a tool, for example, requires a threshold level of dexterity. This can exclude some individuals from using certain objects due to a lack of, or perceived lack of, the required dexterity. This challenge can be exacerbated by many factors, including the size of the objects, the type of engagement between the objects and the tool, and any need to repeatedly engage multiple objects with the tool during a particular procedure or task that utilizes the objects.

Medical fasteners, such as bone screws, staples, and other medical fasteners, are representative of this challenge. For example, bone screws are commonly used in the attachment of bone fixation plates to one or more bones. In these procedures, multiple bone screws of varying sizes, including different widths and lengths, are typically implanted to secure one or more plates to one or more bones. During these procedures, the care provider must engage each screw being implanted onto a driver for subsequent driving into the target bone. This engagement, or loading process, must be performed carefully to avoid dislodgment of the screw from its packaging, which can render it unsuitable for use in the procedure. The loading process must be performed quickly, too, to meet any time demands on the procedure.

Many conventional screw holders position multiple screws in an upright position in reusable trays. This packaging and positioning forces the care provider to use a “stab and grab” loading technique in which the tip of a self-retaining driver is pushed downward into a mating recess of a targeted screw and then retracted vertically, relative to earth, before rotating the driver to a horizontal orientation suitable for the rotation needed to accomplish the desired driving of the screw into the bone. This technique relies solely on the self-retaining feature of the driver to ensure that the screw is not dislodged from the driver before use. Furthermore, the connection between the screw and driver is challenged during rotation of the driver and screw from the initial vertical orientation to a subsequent horizontal orientation that is necessitated by conventional packaging and loading techniques.

A need exists, therefore, for improved holders for objects to be retained, such as screws and other fasteners, kits that include a holder and one or more objects to be retained, and related methods, such as methods of engaging an object with a tool. A particular need exists for improved holders for medical fasteners, such as bone screws, kits including a holder and one or more medical fasteners, and methods of loading a medical fastener onto a tool for implanting the medical fastener into a body of an animal, such as a human being.

### BRIEF SUMMARY OF THE DISCLOSURE AND SELECTED EXAMPLES

Various example holders are described.

An example holder comprises a main body having a first surface and a second surface, the main body defining a retainer having first and second arms, each of the first and second arms comprising a flap of the main body that is movable from a first configuration, in which the respective arm is coplanar with the adjacent portions of the main body, and a second configuration in which the respective arm extends away from the adjacent portions of the main body.

Another example holder comprises a main body having a first surface and a second surface; the main body defining a first retainer having first and second arms, each of the first and second arms comprising a flap of the main body that is movable from a first configuration, in which the respective arm is coplanar with the adjacent portions of the main body, and a second configuration in which the respective arm extends away from the adjacent portions of the main body; and the main body defining a second retainer having third and fourth arms, each of the third and fourth arms comprising a flap of the main body that is movable from a first configuration, in which the respective arm is coplanar with the adjacent portions of the main body, and a second configuration in which the respective arm extends away from the adjacent portions of the main body.

Another example holder comprises a main body having a first surface and a second surface; the main body defining a first set of clips, each clip of the first set of clips comprising a retainer having first and second arms, each of the first and second arms comprising a flap of the main body that is movable from a first configuration, in which the respective arm is coplanar with the adjacent portions of the main body, and a second configuration in which the respective arm extends away from the adjacent portions of the main body; and the main body defining a second set of clips, each clip of the second set of clips comprising a retainer having third, fourth, fifth, and sixth arms, each of the third, fourth, fifth, and sixth arms comprising a flap of the main body that is movable from a first configuration, in which the respective arm is coplanar with the adjacent portions of the main body, and a second configuration in which the respective arm extends away from the adjacent portions of the main body.

Various kits are described.

An example kit comprises a holder according to an embodiment and an object retained on the holder by the retainer.

Another example kit comprises a holder according to an embodiment and a medical fastener retained on the holder by the retainer.

Another example kit comprises a holder according to an embodiment and a bone screw retained on the holder by the retainer.

Another example kit comprises a holder according to an embodiment, the holder having first and second sets of clips, a first set of bone screws retained on the holder by the first set of clips, and a second set of bone screws retained on the holder by the second set of clips.

Various example methods of engaging an object with a tool are described.

Various example methods of loading a medical fastener onto a tool for implanting the medical fastener into a body of an animal, such as a human being, are described.

An example method of loading a medical fastener onto a tool for implanting the medical fastener into a body of an animal comprises inserting a distal end of a driver into a recess defined by a head of a screw retained on a holder according to an embodiment by first and second arms of the retainer; applying a distally directed force on the driver such that the arms of the retainer apply a resistive force on the head of the screw; and applying a distally directed force on the driver such that the head of the screw overcomes the resistive force to release the screw from the holder while remaining secured to the distal end of the driver.

Another example method of loading a medical fastener onto a tool for implanting the medical fastener into a body of an animal comprises inserting a distal end of a driver into a recess defined by a head of a screw retained on a holder according to an embodiment by first and second arms of a first retainer and third and fourth arms of a second retainer; applying a distally directed force on the driver such that the first and second arms of the first retainer apply a resistive force on the head of the screw and the third and fourth arms of the second retainer apply a resistive force on the body of the screw; and applying a distally directed force on the driver such that the head of the screw overcomes the resistive force of the first and second arms of the first retainer and the body of the screw overcomes the resistive force of the third and fourth arms of the second retainer to release the screw from the holder while remaining secured to the distal end of the driver.

Another example method of loading a medical fastener onto a tool for implanting the medical fastener into a body of an animal comprises inserting a distal end of a driver into a recess defined by a head of a screw retained on a holder according to an embodiment by first and second arms of the retainer; applying a distally directed force on the driver such that the arms of the retainer apply a resistive force on the head of the screw; applying a distally directed force on the driver such that the head of the screw overcomes the resistive force to release the screw from the holder while remaining secured to the distal end of the driver; inserting a distal end of the driver into a recess defined by a head of a second screw retained on the holder by third and fourth arms of a second retainer and fifth and sixth arms of a third retainer; applying a distally directed force on the driver such that the third and fourth arms of the second retainer apply a resistive force on the head of the second screw and the fifth and sixth arms of the third retainer apply a resistive force on the body of the second screw; and applying a distally directed force on the driver such that the head of the second screw overcomes the resistive force of the third and fourth arms of the second retainer and the body of the second screw overcomes the resistive force of the fifth and sixth arms of the third retainer to release the second screw from the holder while remaining secured to the distal end of the driver.

Additional understanding of the inventive holders, kits, and methods can be obtained by reviewing the detailed description of selected examples, below, and the referenced drawings.

### BRIEF DESCRIPTION OF THE FIGURES

FIG. 1 is a top view of an example holder.

FIG. 2 is a perspective view of an example kit that includes the example holder illustrated in FIG. 1 and a bone screw.

FIG. 3 is a top view of another example holder.

FIG. 4 is a perspective view of another example kit that includes the example holder illustrated in FIG. 3 and a bone screw.

FIG. 5 is a perspective view of another example kit that includes another example holder and multiple bone screws. The holder is shown in a first configuration.

FIG. 6 is a front view of the example kit illustrated in FIG. 5. The holder is shown in a second configuration.

FIG. 7 is a perspective view of another example kit that includes another example holder and multiple bone screws. The kit is show in a stage of use in a method according to an embodiment.

FIG. 8 is a perspective view of the example kit illustrated in FIG. 7. The kit is show in another stage of use in a method according to an embodiment.

FIG. 9 is a top view of another example holder.

FIG. 10 is a perspective view of an example kit that includes the example holder illustrated in FIG. 9 and a medical staple.

FIG. 11 is a top view of another example holder.

FIG. 12 is a perspective view of an example kit that includes the example holder illustrated in FIG. 11 and a medical staple.

FIG. 13 is a flowchart representation of an example method of loading a medical fastener onto a tool for implanting the medical fastener into a body of an animal.

FIG. 14 is a flowchart representation of another example method of loading a medical fastener onto a tool for implanting the medical fastener into a body of an animal.

FIG. 15 is a flowchart representation of another example method of loading a medical fastener onto a tool for implanting the medical fastener into a body of an animal.

FIG. 16 is a flowchart representation of an example method of loading multiple medical fasteners onto a tool for implanting the medical fasteners into a body of an animal.

### DETAILED DESCRIPTION OF SELECTED EXAMPLES

The following detailed description and the appended drawings describe and illustrate various example holders for objects to be retained, such as screws and other fasteners, kits that include a holder and one or more objects to be retained, and related methods, such as methods of loading a medical fastener onto a tool for implanting the medical fastener into a body of an animal. The description and illustration of these examples enable one skilled in the art to make and use examples of the inventive holders and kits and to perform examples of the related inventive methods. The selected examples are representative in nature; their inclusion in this detailed description, and the exclusion of other examples, does not limit the scope of the claims in any manner.

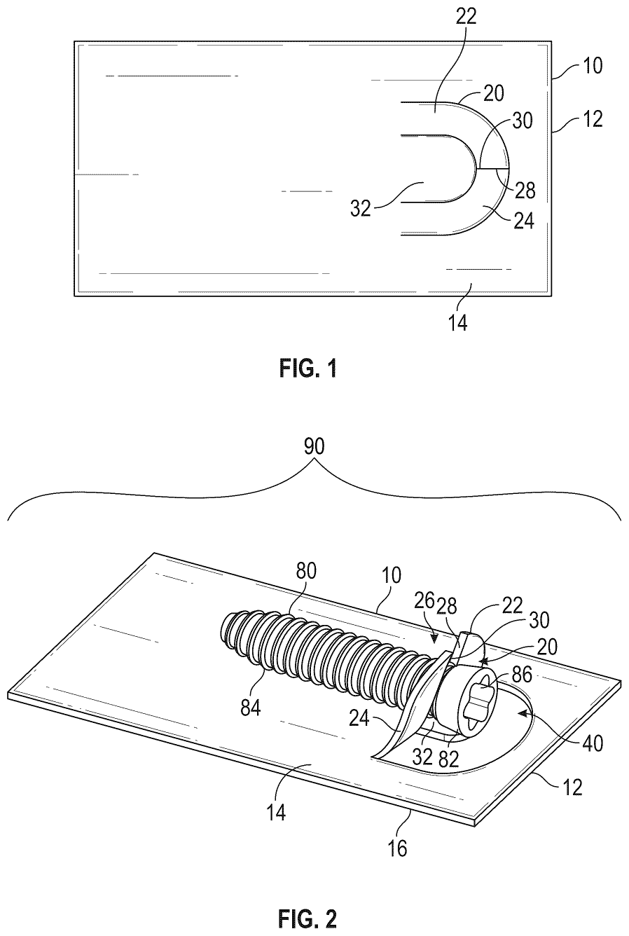

FIG. 1 illustrates a first example holder 10 for an object to be retained. As described in detail below, the holder 10 is suitable to retaining a medical fastener, such as a bone screw. FIG. 2 illustrates a first example kit 90, which includes holder 10 and bone screw 80 retained on holder 10.

Holder 10 includes a main body 12 having a first surface 14 and a second surface 16. As best illustrated in FIG. 2, the main body 12 has a thickness extending between the first surface 14 and second surface 16. In this example, main body 12 is a flat, planar body having a rectangular configuration. While the entire main body need not form a flat, planar body, inclusion of at least a portion of a main body that provides a flat, planar portion for formation and storage of retainers, as described in more detail below, is considered advantageous at least because it provides efficient storage and performance of holders, and kits, according to embodiments.

Main body 12 defines retainer 20 that includes first 22 and second 24 arms. Each of the first 22 and second 24 arms comprises a flap of the material of the main body 12 that is movable from a first configuration, in which the respective arm 22, 24 is coplanar with the adjacent portions of the main body 12, as illustrated in FIG. 1, and a second configuration in which the respective arm 22, 24 extends away from the adjacent portions of the main body 12, as illustrated in FIG. 2. The retainer 20, with arms 22, 24 movable in this manner, also is movable between a first configuration in which both arms 22, 24 are coplanar with the adjacent portions of the main body 12, illustrated in FIG. 1, and a second configuration in which both arms 22, 24 extend away from the adjacent portions of the main body 12, as illustrated in FIG. 2. Recess 40 is formed in main body 12 when the retainer 20 is in the second configuration and represents the void of material left by movement of retainer 20 from the coplanar configuration of its first configuration. In the illustrated embodiment, recess 40 extends through the entire thickness of the main body 12. It is noted, though, that recess can extend into only a portion of the thickness of the main body 12.

The first 22 and second 24 arms are separated by channel 26 extending between a terminal surface 28 of the first arm 22 and an opposing terminal surface 30 of the second arm 24. Inclusion of channel 26 enables the first 22 and second 24 arms to move between their respective first and second configurations independently of each other. As best illustrated in FIG. 1, when retainer 20 is in its first configuration, with each arm 22, 24 in its first configuration, terminal surfaces 28, 30 oppose each other and, in this example, contact each other. In this configuration, channel 26 is minimized and, in this example, essentially comprises a cut line between the surfaces 28, 30. Also in this configuration, first 22 and second 24 arms form a stilted arch that is coplanar with main body 12. This structural arrangement is considered particularly advantageous at least because it provides desirable retention of screw 80 and provides arms 22, 24 of sufficient height relative to main body 12, when in the respective second configuration, to accommodate a variety of screws. As best illustrated in FIG. 2, when retainer 20 is in its second configuration, with each arm 22, 24 in its second configuration, terminal surfaces 28, 30 still oppose each other but channel 26 has a relatively larger width extending between the terminal surfaces 28, 30.

In the second configuration, channel 26 can receive a portion of a retained object, such as bone screw 80. For example, as illustrated in FIG. 2, bone screw 80 can be passed into channel 26 such that head 82 of screw 80 is disposed on the side of retainer 20 in which recess 40 is formed in the main body 12 while the body 84 of screw 80 is disposed on the other, opposite side of retainer 20. In this configuration, the first 22 and second 24 arms of retainer 20 contact head 82 of screw and provide a resistive force against movement of head 82 of screw 80 in a direction away from recess 40, such as by a pushing force applied to a tool engaged with recess 86 of screw 80, such as a driver. In this manner, retainer 20 resists movement of screw 80 along first surface 14 of main body 12 of holder 10 and generally away from recess 40, effectively retaining screw 80 on main body 12 until sufficient force is applied to screw 80 to overcome this resistance to movement. Also in this configuration, head 82 rests on projection 32 of main body 12 that remains coplanar with adjacent portions of main body 12 and is disposed between arms 22, 24. Accordingly, screw 80 can be released from holder 10 by application of a distally directed force, generally along a plane parallel to surface 14 and to the left in FIG. 2, an upward directed force, generally perpendicular to surface 14, or a force that includes both a distally directed component and an upward component. As such, holder 10 is well-suited for users to release screw 10 by any approach they prefer without sacrificing the retention capability of holder 10.

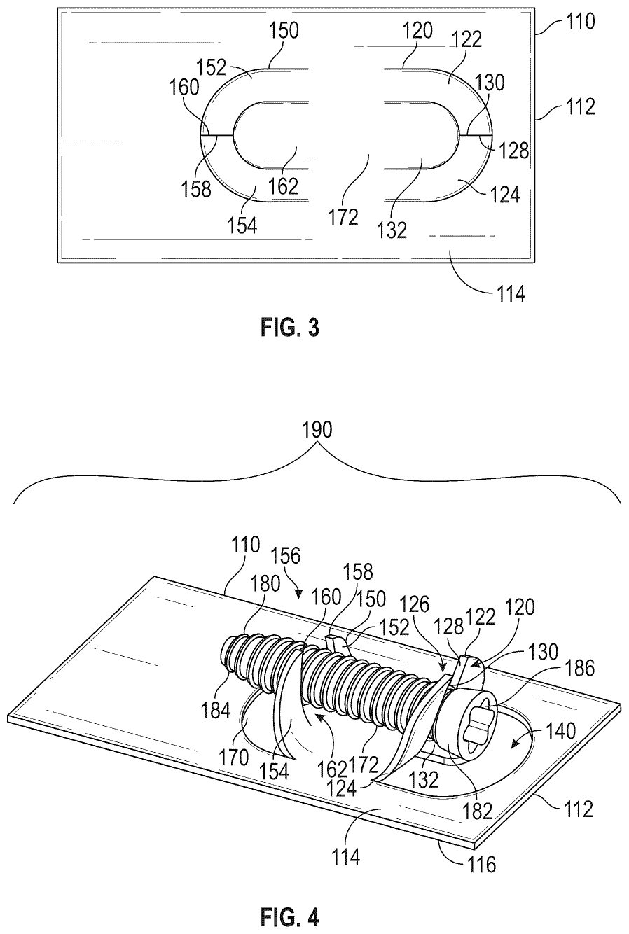

FIG. 3 illustrates another example holder 110 for an object to be retained. As described in detail below, the holder 110 is suitable to retaining a medical fastener, such as a bone screw. FIG. 4 illustrates another example kit 190, which includes holder 110 and bone screw 180 retained on holder 110. Holder 110 is similar to holder 10 illustrated in FIGS. 1 and 2 and described above, except as detailed below. Reference numbers in FIGS. 3 and 4 that reference structure that correlates to structure from holder 10 has the same reference number, incremented by 100.

Holder 110 includes a main body 112 having a first surface 114, a second surface 116, and a thickness extending between the first surface 114 and second surface 116. Main body 112 defines first retainer 120 that includes first 122 and second 124 arms. Each of the first 122 and second 124 arms comprises a flap of the material of the main body 112 that is movable from a first configuration, in which the respective arm 122, 124 is coplanar with the adjacent portions of the main body 112, as illustrated in FIG. 3, and a second configuration in which the respective arm 122, 124 extends away from the adjacent portions of the main body 112, as illustrated in FIG. 4. The first retainer 120, with arms 122, 124 movable in this manner, also is movable between a first configuration in which both arms 122, 124 are coplanar with the adjacent portions of the main body 112, illustrated in FIG. 3, and a second configuration in which both arms 122, 124 extend away from the adjacent portions of the main body 112, as illustrated in FIG. 4. Recess 140 is formed in main body 112 when the first retainer 120 is in its second configuration and represents the void of material left by movement of first retainer 120 from the coplanar configuration of its first configuration. The first 122 and second 124 arms are separated by channel 126 extending between a terminal surface 128 of the first arm 122 and an opposing terminal surface 130 of the second arm 124.

In this example, holder 110 includes second retainer 150 that includes third 152 and fourth 154 arms. Similar to the first 122 and second 124 arms of the first retainer 120, each of the third 152 and fourth 154 arms of the second retainer 150 comprises a flap of the material of the main body 112 that is movable from a first configuration, in which the respective arm 152, 154 is coplanar with the adjacent portions of the main body 112, as illustrated in FIG. 3, and a second configuration in which the respective arm 152, 154 extends away from the adjacent portions of the main body 112, as illustrated in FIG. 4. The second retainer 150, with arms 152, 154 movable in this manner, also is movable between a first configuration in which both arms 152, 154 are coplanar with the adjacent portions of the main body 112, illustrated in FIG. 3, and a second configuration in which both arms 152, 154 extend away from the adjacent portions of the main body 112, as illustrated in FIG. 4. Recess 170 is formed in main body 112 when the second retainer 150 is in its second configuration and represents the void of material left by movement of second retainer 150 from the coplanar configuration of its first configuration. The third 152 and fourth 154 arms are separated by channel 156 extending between a terminal surface 158 of the third arm 152 and an opposing terminal surface 160 of the fourth arm 154.

In this example, first 122 and second 124 arms form a first stilted arch that is coplanar with main body 112 and third 152 and fourth 154 arms form a second stilted arch that is coplanar with main body 112. This structural arrangement is considered particularly advantageous at least because it provides desirable retention of screw 180 and provides arms 122, 124, 152, 154 of sufficient height relative to main body 12, when in the respective second configuration, to accommodate a variety of screws.

As best illustrated in FIG. 3, when first retainer 120 is in its first configuration, terminal surfaces 128, 130 oppose each other and, in this example, contact each other to minimize the width of channel 126. As best illustrated in FIG. 4, when first retainer 120 is in its second configuration, with each arm 122, 124 in its second configuration, terminal surfaces 128, 130 still oppose each other but channel 126 has a relatively larger width extending between the terminal surfaces 128, 130. Similarly, channel 156 between opposing surfaces 158, 160 of arms 152, 154 of the second retainer 150 is minimized when contacting each other with second retainer 150, and arms 152, 154, in the first configuration, illustrated in FIG. 3. In the second configuration, illustrated in FIG. 4, terminal surface 158, 160 still oppose each other but channel 156 has a relatively larger width extending between terminal surface 158, 160.

In the second configuration, each of the channels 126, 156 can receive a portion of a retained object, such as bone screw 180. For example, as illustrated in FIG. 4, bone screw 180 can be passed into channel 126 such that head 182 of screw 180 is disposed on the side of first retainer 120 in which recess 140 is formed in the main body 112 while the body 184 of screw 180 is disposed on the other, opposite side of the first retainer 120. Also, another portion of the body 184 of screw 180 is passed into channel 156, leaving a distal portion 188 of screw 180 to extend over recess 170 formed in main body 112. In this configuration, the first 122 and second 124 arms of the first retainer 120 contact head 82 of screw and provide a resistive force against movement of head 182 of screw 180 away from recess 140 and toward recess 170, such as by a pushing force applied to a tool engaged in recess 186 of screw 180, such as a driver. Third 152 and fourth 154 arms of second retainer 150 contact body 184 of screw and also provide a resistive force against such movement. In this manner, first retainer 120 and second retainer 150 resist movement of screw 180 along first surface 114 of main body 112 of holder 110 and generally away from recess 140, effectively retaining screw 180 on main body 112 until sufficient force is applied to screw 180 to overcome this resistance to movement. Also in this configuration, head 182 of screw 180 rests on projection 132 of main body 112 that remains coplanar with adjacent portions of main body 112 and is disposed between arms 122, 124 of the first retainer 120.

While example screw 180 has an axial length that positions distal portion 188, which is at the opposite end of screw 180 from head 182, across recess 170, it is noted that screws having a shorter axial length can be used with holder 110, including screws having an axial length that positions a distal portion, including a distal end, axially within the boundaries of recess 170. Advantageously, a screw in a kit having a holder with first and second retainers, such as holder 110 with first 120 and second 150 retainers, has an axial length that is sufficiently long to position the distal end of the screw axially beyond the first and second arms of the second retainer, such as first 158 and second 160 arms of second retainer 150, when the screw is positioned in the first retainer, such as in first retainer 120 as described above. It is noted, though, that shorter screws can be included in a kit having a holder with first and second retainers. In these examples, the second retainer can be left coplanar with the main body of the holder.

In the example holder 110 illustrated in FIG. 3 and FIG. 4, the second retainer 150 extends away from main body 112 in a direction that is different from the direction at which the first retainer 120 extends away from the main body. As best illustrated in FIG. 4, this places the recess 170 left by arms 152, 154 opposite the recess 140 left by arms 122, 124 with a portion of main body 172 remaining between the first retainer 120 and the second retainer 150. In other embodiments that include a second retainer, though, the second retainer can have the opposite orientation than that illustrated in FIGS. 3 and 4. Specifically, in the second configuration, the second retainer can extend away from the main body in the same direction in which the first retainer extends away from the main body. This structural arrangement places the recess left by the second retainer adjacent the recess left by the first retainer. The illustrated structural arrangement is considered advantageous, though, at least because the first 120 and second 150 retainers provide different levels of resistive force as the screw 180 is removed from the holder 110.

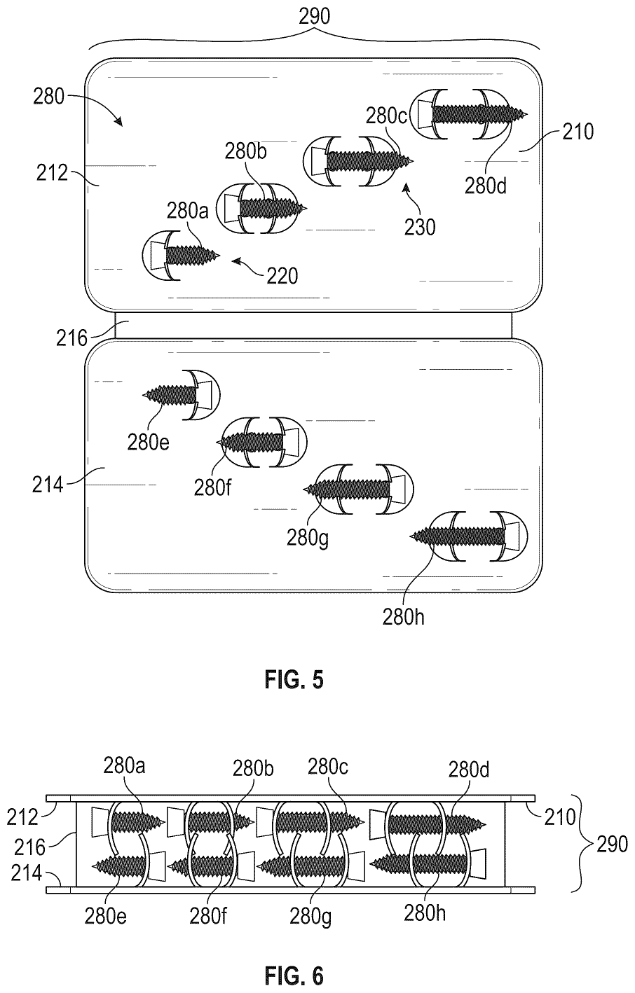

FIGS. 5 and 6 illustrate another example kit 290. Kit 290 includes example holder 210 and a plurality of screws 280. In this example, holder 210 includes first 212 and second 214 panels joined by connector 216. In the illustrated embodiment, the first 212 and second panels 214, and connector 216, are integrally formed as a continuous section of material, but these elements could comprise separate structures joined together. Holder 210 includes first 220 and second 230 sets of clips. Each clip of the first set of clips 220 is similar to the retainer 20 of the holder 10 illustrated in FIGS. 1 and 2, including only first and second arms. Each clip of the second set of clips 230 is similar to the first 120 and second 150 retainers of the holder 110 illustrated in FIGS. 3 and 4, including first, second, third, and fourth arms. Each set of clips 220, 230 can include any suitable number of clips, such as one, two, a plurality, three, four, five, six, seven, eight, nine, ten, more than ten, a dozen, or more clips. A skilled artisan will be able to select an appropriate number of clips for each set of clips in a kit according to a particular embodiment based on various considerations, such as the number, dimensions, such as axial length, and structural configuration of the screws in the kit. In the illustrated example kit, first set of clips 220 includes two holders, one for each of two screws 280a, 280e having the shortest axial length among all screws in the plurality of screws 280. The second set of clips 230 includes six clips, which is equal to the total number of screw in the plurality of screws less the total number of screws having the shortest axial length among all screws in the plurality of screws 280.

Holder 210 is movable between a first configuration, illustrated in FIG. 5, in which first 212 and second 214 panels are coplanar, and a second configuration, illustrated in FIG. 6, in which holder 210 is folded along connector 216 to position first panel 212 directly above second panel 214. This configuration is considered advantageous because it provides the first configuration, in which all screws of the plurality of screws 280 are accessible for engagement with a tool, and second configuration, in which each panel 212, 214 covers the screws of the plurality of screws 280 that are retained by clips of the other panel 212, 214, reducing the possibility of an unintended disruption in the retention of such screws by the clips of the other panel 212, 214.

The plurality of screws 280 includes individual screws 280a, 280b, 280c, 280d, 280e, 280f, 280g, 280h of varying sizes. While any screw can be associated with any clip of either set of clips 220, 230, it is considered advantageous to associate the relatively smaller screws with clips of the first set of clips 220 and relatively larger screws with clips of the second set of clips 230. In one example, screws having an axial length equal to or less than 10 mm, such as 10 mm and 8 mm length screws, are associated with clips of a first set of clips 220 and screws having an axial length greater than 10 mm, such as 12 mm, 14 mm, and greater lengths, are associated with clips of the second set of clips 230.

The structural arrangement of the holder 210 illustrated in FIGS. 5 and 6 is considered advantageous at least because it provides a book-shaped rectangular card that can carry a plurality of screws of varying sizes, such as the illustrated screws 280a, 280b, 280c, 280d, 280e, 280f, 280g, 280h, in a relatively small form factor with operational properties that are readily understood by care providers. Also, the structural arrangement of holder 210 provides efficient and cost effective packaging as compared to conventional individual screw packaging, and the holder 210 can easily be used in methods according to embodiments. Also, in the illustrated example, the plurality of screws 280 includes a first set of screws 280a, 280b, 280c, 280d retained on holder 210 by clips on first panel 12 and a second set of screws 280e, 280f, 280g, 280h retained on holder 210 by clips on second panel 14. Screws 280a, 280b, 280c, 280d of the first set of screws are all arranged in the respective clips with their respective heads on the same side of the respective clip (the left side in the Figure), while screws 280e, 280f, 280g, 280h of the second set of screws are all arranged in the respective clips with their respective heads on the opposite side of the respective clip (the right side in the Figure). This arrangement is considered advantageous at least because it provides a staggered arrangement when the holder is in a folded configuration, such as second configuration illustrated in FIG. 6. It is noted, though, that other arrangements of screws may be desirable. For example, while the illustrated arrangement may be advantageous from a packaging perspective, it does require advancement of a tool from different directions to engage screws of the first and second sets. An arrangement in which all screws are positioned in their respective clips in the same orientation allows engagement of all screws by advancing a tool in the same direction, which may be desirable.

The main body of the holder in all embodiments can be made of any suitable material. The material need only be biocompatible, or be able to be rendered biocompatible, and be able to provide a resistive force against movement of a screw disposed on the holder as described herein. The inventors have determined that flexible plastics, including low density and high density materials, are advantageous materials for forming a main body of a holder according to an embodiment. High density polyethylene (HDPE) is considered particularly advantageous at least because of its well-characterized nature, ready availability, and ease of handling during formation and cutting. Furthermore, the retainers in all embodiments are preferably formed as cuts through the entire thickness of the material, such as with a die. Flexible plastics are also considered advantageous because when cut in this manner, and the flaps of the arms of the retainers are moved away from the main body, such as when moving the arms of a retainer to a second configuration of a retainer, are biased toward returning to the first orientation in which the arms are coplanar with adjacent portions of the main body. In this manner, flexible plastic materials provide are considered advantageous at least because they can be easily cut to provide collapsible and biased retainers for holding one or more bone screws and for providing a resistive force on a portion of the one or more bone screws during loading onto a driver, such as during performance of a method of an embodiment.

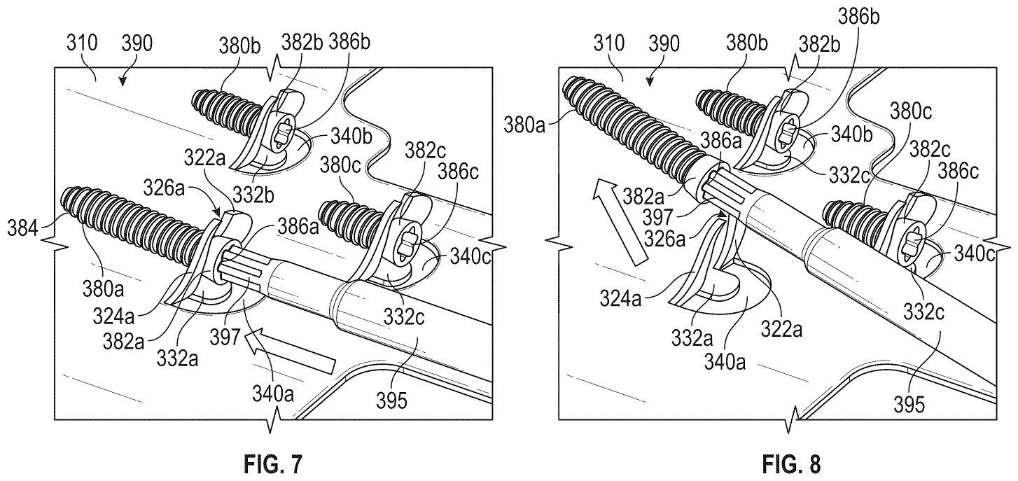

FIGS. 7 and 8 illustrate another example kit 390 that includes another example holder 310 and multiple bone screws 380a, 380b, 380c. Holder 310 includes first retainer 320a that retains first screw 380a, second retainer 320b that retains second screw 380b, and third retainer 320c that retains third screw 380c. Each retainer 320a, 320b, 320c is similar to retainer 20 described above and illustrated in FIGS. 1 and 2. Reference numbers in FIGS. 7 and 8 that reference structure that correlates to structure from holder 10 has the same reference number, incremented by 300. FIG. 7 illustrates the kit 390 in a stage of use in a method according to an embodiment, as described in detail below. In this stage, distal end 397 of driver 395 is inserted into recess of screw 380a such that screw 380a is engaged with driver 395. Arms 322a and 324a are exerting a resistive force against movement of screw 380a in the direction of the arrow. FIG. 8 illustrates the kit 390 in another stage of use in a method to an embodiment. In this stage, distal end 397 of driver 395 has been pushed through channel 326a, overcoming the resistive force of arms 322a, 324a, freeing screw 380a from arms 322a, 324a and holder 310.

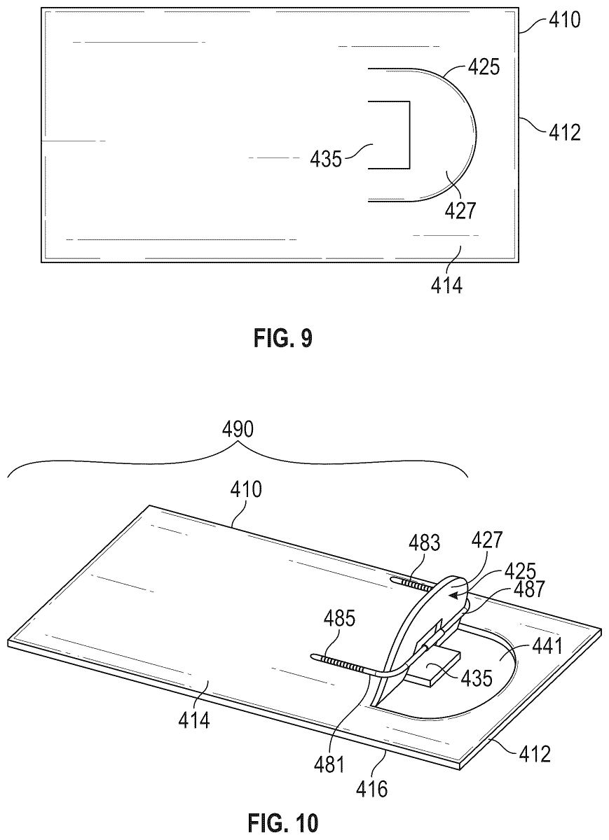

FIG. 9 illustrates another example holder 410 for an object to be retained. As described in detail below, the holder 410 is suitable to retaining a medical fastener, such as a medical staple. FIG. 10 illustrates another example kit 490, which includes holder 410 and medical staple 481 retained on holder 410. Holder 410 is similar to holder 10 illustrated in FIGS. 1 and 2 and described above, except as detailed below. Reference numbers in FIGS. 9 and 10 that reference structure that correlates to structure from holder 10 has the same reference number, incremented by 400.

Holder 410 includes a main body 412 having a first surface 414, a second surface 416, and a thickness extending between the first surface 414 and second surface 416. Main body 412 defines retainer 425. In this embodiment, retainer 425 comprises a continuous flap 427 of the material of the main body 412 that is movable from a first configuration, in which the flap is coplanar with the adjacent portions of the main body 412, as illustrated in FIG. 9, and a second configuration in which the flap 427 extends away from the adjacent portions of the main body 412, as illustrated in FIG. 10. The retainer 425, with flap 427 movable in this manner, also is movable between a first configuration in which the flap 427 is coplanar with the adjacent portions of the main body 412, illustrated in FIG. 9, and a second configuration in which the flap 427 extends away from the adjacent portions of the main body 412, as illustrated in FIG. 10. Recess 441 is formed in main body 112 when the retainer 425 is in its second configuration and represents the void of material left by movement of retainer 425 from the coplanar configuration of its first configuration. In this embodiment, the flap 427 is continuous and does not include any channel or other separation in either configuration.

In this example, flap 427 defines an outer arcuate edge 429 and a series 431 of inner linear edges 431 that cooperatively define a rectangular opening when flap 427 is in the second configuration, as illustrated in FIG. 10. This structural arrangement defines projection 435 having linear edges when flap 427 is in its second configuration. Medical staple 481 includes first 483 and second 485 legs and body 487 extending between legs 481, 483. A method of engaging an object with a tool includes an initial step of inserting a distal end of a driver into a recess defined by a portion of an object retained on a holder according to an embodiment. Another step comprises applying a distally directed force on the driver such that the retainer of the holder applies a resistive force on a portion of the object. Another step comprises applying a distally directed force on the driver sufficient to overcome the resistive force to release the object from the holder while remaining secured to the distal end of the driver.

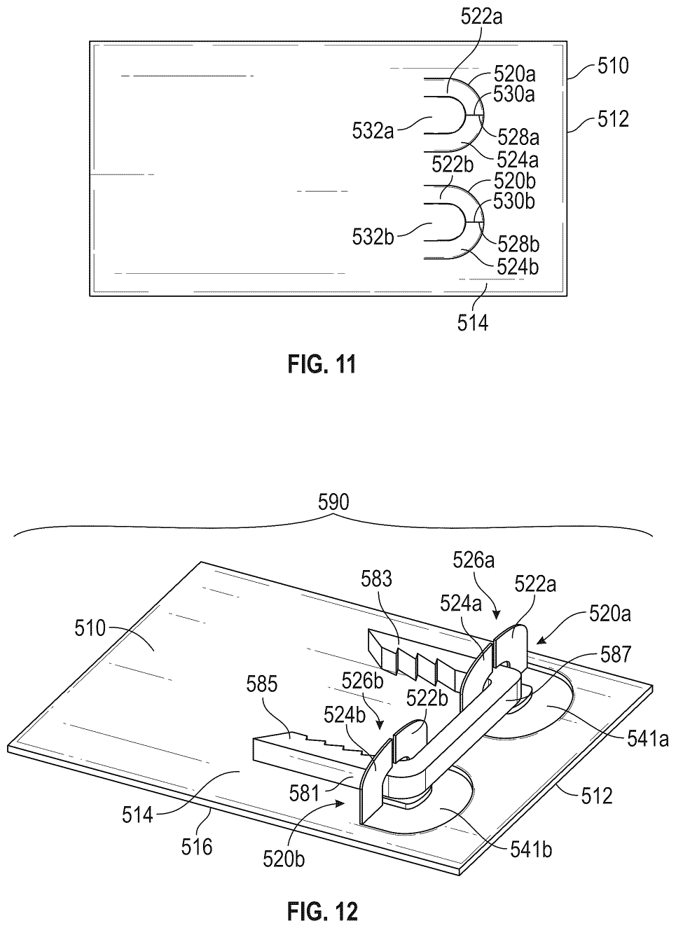

FIG. 11 illustrates another example holder 510 for an object to be retained. As described in detail below, the holder 510 is suitable to retaining a medical fastener, such as a medical staple. FIG. 12 illustrates another example kit 590, which includes holder 510 and medical staple 581 retained on holder 510. Holder 410 is similar to holder 10 illustrated in FIGS. 1 and 2 and described above, except as detailed below. Reference numbers in FIGS. 11 and 12 that reference structure that correlates to structure from holder 10 has the same reference number, incremented by 500.

Holder 510 includes a main body 512 having a first surface 514, a second surface 516, and a thickness extending between the first surface 514 and second surface 516. In this embodiment, main body 512 defines first retainer 520a and second retainer 520b. First retainer 520a has first 522a and second 524a arms, each of which comprises a flap of the material of the main body 512 that is movable from a first configuration, in which the respective arm 522a, 524a is coplanar with the adjacent portions of the main body 512, as illustrated in FIG. 11, and a second configuration in which the respective arm 522a, 524a extends away from the adjacent portions of the main body 512, as illustrated in FIG. 12. The first retainer 520a, with arms 522a, 524a movable in this manner, also is movable between a first configuration in which both arms 522a, 524a are coplanar with the adjacent portions of the main body 512, illustrated in FIG. 11, and a second configuration in which both arms 522a, 524a extend away from the adjacent portions of the main body 512, as illustrated in FIG. 12. A first recess 541a is formed in main body 512 when the first retainer 520a is in the second configuration and represents the void of material left by movement of the first retainer 520a from the coplanar configuration of its first configuration. The first 522a and second 524a arms are separated by channel 526a extending between a terminal surface 528a of the first arm 522a and an opposing terminal surface 530a of the second arm 524a.

Second retainer 520b has first 522b and second 524b arms, each of which comprises a flap of the material of the main body 512 that is movable from a first configuration, in which the respective arm 522b, 524b is coplanar with the adjacent portions of the main body 512, as illustrated in FIG. 11, and a second configuration in which the respective arm 522a, 524b extends away from the adjacent portions of the main body 512, as illustrated in FIG. 12. The second retainer 520b, with arms 522b, 524b movable in this manner, also is movable between a first configuration in which both arms 522b, 524b are coplanar with the adjacent portions of the main body 512, illustrated in FIG. 11, and a second configuration in which both arms 522b, 524b extend away from the adjacent portions of the main body 512, as illustrated in FIG. 12. A second recess 541b is formed in main body 512 when the second retainer 520b is in the second configuration and represents the void of material left by movement of the second retainer 520b from the coplanar configuration of its first configuration. The first 522b and second 524b arms are separated by channel 526b extending between a terminal surface 528b of the first arm 522b and an opposing terminal surface 530b of the second arm 524b.

Inclusion of channels 526a, 526b enables the first 522a, 522b and second 524a, 524b arms of each retainer 520a, 520b to move between their respective first and second configurations independently of each other. As best illustrated in FIG. 11, when first 520a is in its first configuration, terminal surfaces 528a, 530a of the first retainer 520a oppose each other and, in this example, contact each other. Similarly, when second 520b is in its first configuration, terminal surfaces 528b, 530b of the second retainer 520b oppose each other and, in this example, contact each other.

In this example, as best illustrated in FIG. 12, holder 510 includes two retainers 520a, 520b that each engage the same object to be retained on the holder, which, in this example, is medical staple 581. Medical staple includes first 583 and second 585 legs and body 587 extending between legs 581, 583. Inclusion of first 520a and second 520b retainers, with the structure described above, is considered advantageous at least because it permits release of medical staple 581 from holder 510 by application of a distally directed force, generally along a plane parallel to surface 514 and to the left in FIG. 12, an upward directed force, generally perpendicular to surface 514, or a force that includes both a distally directed component and an upward component. As such, holder 510 is well-suited for users to release medical staple 581 by any approach they prefer without sacrificing the retention capability of holder 510.

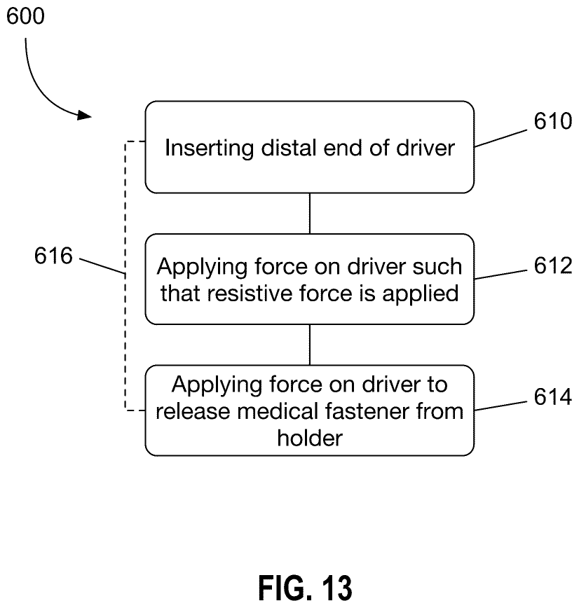

FIG. 13 illustrates an example method 600 of loading a medical fastener onto a tool for implanting the medical fastener into a body of an animal, such as a human being. An initial step 610 comprises engaging a medical fastener retained on a holder according to an embodiment with a distal end of a tool adapted for implanting the medical fastener into a body. In a particular example, the medical fastener comprises a bone screw having a head that defines the recess and the tool comprises a driver having a distal end sized and configured to be inserted into the recess to engage the bone screw. Another step 612 comprises applying a force on the driver such that the retainer of the holder applies a resistive force on a portion of the medical fastener. This step can be performed by applying a distally directed force generally along a plane parallel to the surface of the holder, applying an upward directed force generally perpendicular to surface of the holder, or applying a force that includes both a distally directed component and an upward component. In a particular example, this step is performed such that the retainer of the holder applies a resistive force on the head of the bone screw. Another step 614 comprises applying a force on the driver sufficient to overcome the resistive force to release the medical fastener from the holder while remaining secured to the distal end of the driver. This step can be performed by applying a distally directed force generally along a plane parallel to the surface of the holder, applying an upward directed force generally perpendicular to surface of the holder, or applying a force that includes both a distally directed component and an upward component. In a particular example, this step is performed such that the bone screw is released from the holder while remaining secured to the distal end of the driver. As represented by optional step 616, steps 610, 612, and 614 can be repeated with another medical fastener retained on the holder by another retainer, up to a total number of repetitions for the total number of medical fasteners retained on the holder. Also, steps 612 and 614 can be performed as one continuous action.

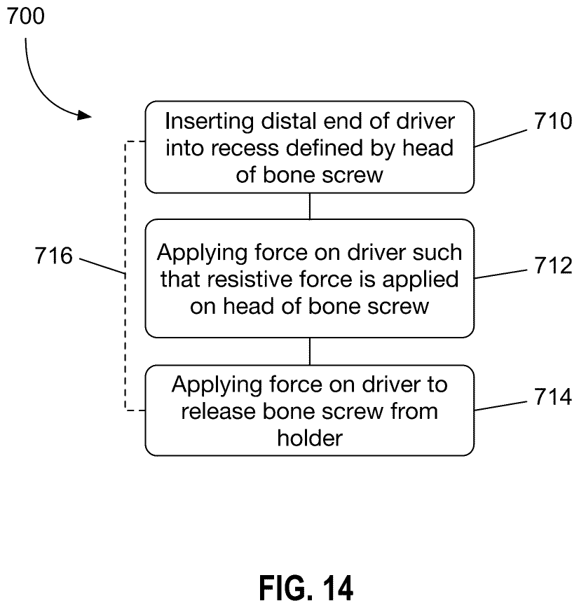

FIG. 14 illustrates an example method 700 of engaging a bone screw with a driver for implanting the bone screw into a body of an animal, such as a human being. An initial step 710 comprises inserting a distal end of a driver into a recess defined by a head of a bone screw retained on a holder according to an embodiment by first and second arms of a retainer defined by the holder. Another step 712 comprises applying a force on the driver such that the first and second arms of the retainer apply a resistive force on the head of the screw. This step can be performed by applying a distally directed force generally along a plane parallel to the surface of the holder, applying an upward directed force generally perpendicular to surface of the holder, or applying a force that includes both a distally directed component and an upward component. Another step 714 comprises applying a force on the driver such that the head of the screw overcomes the resistive force of the first and second arms of the retainer to release the screw from the holder while remaining secured to the distal end of the driver. This step can be performed by applying a distally directed force generally along a plane parallel to the surface of the holder, applying an upward directed force generally perpendicular to surface of the holder, or applying a force that includes both a distally directed component and an upward component. Also, steps 712 and 714 can be performed as one continuous action. As represented by optional step 716, steps 710, 712, and 714 can be repeated with another bone screw retained on the holder by another retainer, up to a total number of repetitions for the total number of bone screws retained on the holder.

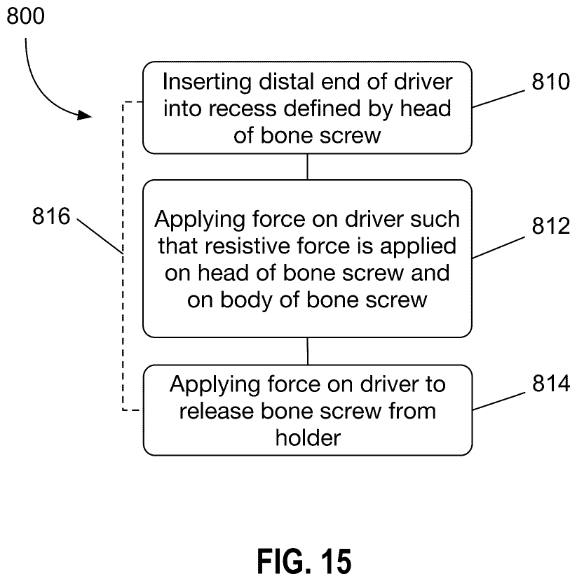

FIG. 15 illustrates an example method 800 of engaging a bone screw with a driver for implanting the bone screw into a body of an animal, such as a human being. An initial step 810 comprises inserting a distal end of a driver into a recess defined by a head of a bone screw retained on a holder according to an embodiment by first and second arms of a first retainer defined by the holder and third and fourth arms of a second retainer defined by the holder. Another step 812 comprises applying a force on the driver such that the first and second arms of the first retainer apply a resistive force on the head of the screw and the third and fourth arms of the second retainer apply a resistive force on the body of the screw. This step can be performed by applying a distally directed force generally along a plane parallel to the surface of the holder, applying an upward directed force generally perpendicular to surface of the holder, or applying a force that includes both a distally directed component and an upward component. Another step 814 comprises applying a force on the driver such that the head of the screw overcomes the resistive force of the first and second arms of the first retainer and the body of the screw overcomes the resistive force of the third and fourth arms of the second retainer to release the screw from the holder while remaining secured to the distal end of the driver. This step can be performed by applying a distally directed force generally along a plane parallel to the surface of the holder, applying an upward directed force generally perpendicular to surface of the holder, or applying a force that includes both a distally directed component and an upward component. Also, steps 812 and 814 can be performed as one continuous action. As represented by optional step 816, steps 810, 812, and 814 can be repeated with another bone screw retained on the holder by another retainer, up to a total number of repetitions for the total number of bone screws retained on the holder.

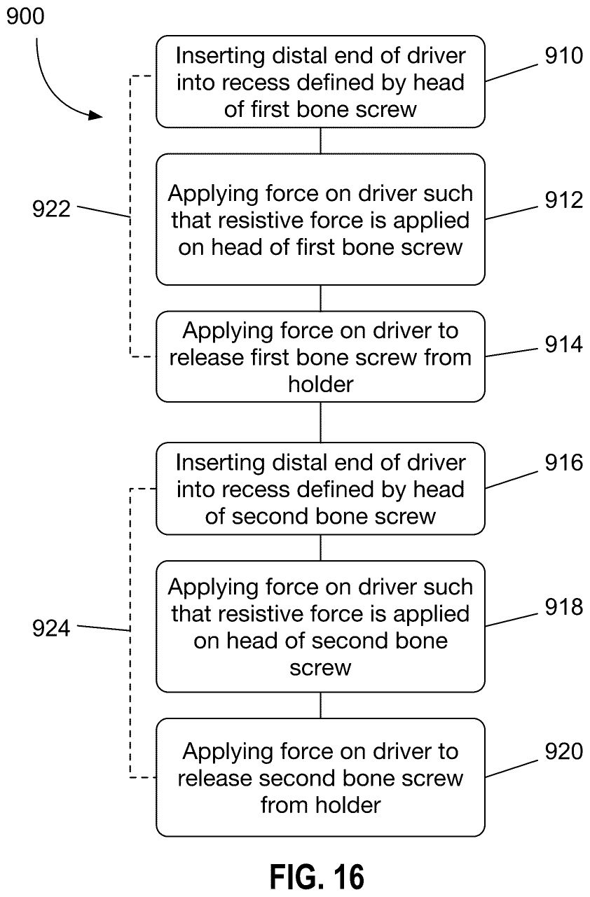

FIG. 16 illustrates an example method 900 of engaging multiple bone screws with a driver for implanting the bone screws into a body of an animal, such as a human being. An initial step 910 comprises inserting a distal end of a driver into a recess defined by a head of a first bone screw retained on a holder according to an embodiment by first and second arms defined by the holder. Another step 912 comprises applying a force on the driver such that the arms of the retainer apply a resistive force on the head of the first bone screw. This step can be performed by applying a distally directed force generally along a plane parallel to the surface of the holder, applying an upward directed force generally perpendicular to surface of the holder, or applying a force that includes both a distally directed component and an upward component. Another step 914 comprises applying a distally directed force on the driver sufficient to overcome the resistive force and release the screw from the holder while remaining secured to the distal end of the driver. This step can be performed by applying a distally directed force generally along a plane parallel to the surface of the holder, applying an upward directed force generally perpendicular to surface of the holder, or applying a force that includes both a distally directed component and an upward component. Also, steps 912 and 914 can be performed as one continuous action. Another step 916 comprises inserting a distal end of the driver into a recess defined by a head of a second bone screw retained on the holder by third and fourth arms of a second retainer defined by the holder and fifth and sixth arms of a third retainer defined by the holder. Another step 918 comprises applying a force on the driver such that the third and fourth arms of the second retainer apply a resistive force on the head of the second screw and the fifth and sixth arms of the third retainer apply a resistive force on the body of the second screw. This step can be performed by applying a distally directed force generally along a plane parallel to the surface of the holder, applying an upward directed force generally perpendicular to surface of the holder, or applying a force that includes both a distally directed component and an upward component. Another step 920 comprises applying a force on the driver such that the head of the second screw overcomes the resistive force of the third and fourth arms of the second retainer and the body of the second screw overcomes the resistive force of the fifth and sixth arms of the third retainer to release the second screw from the holder while remaining secured to the distal end of the driver. This step can be performed by applying a distally directed force generally along a plane parallel to the surface of the holder, applying an upward directed force generally perpendicular to surface of the holder, or applying a force that includes both a distally directed component and an upward component. Also, steps 918 and 920 can be performed as one continuous action. As represented by optional step 922, steps 910, 912, and 914 can be repeated with another bone screw retained on the holder by a single retainer, up to a total number of repetitions for the total number of bone screws retained on the holder by a single retainer. Also, as represented by optional step 924, steps 916, 918, and 920 can be repeated with another bone screw retained on the holder by two retainers, up to a total number of repetitions for the total number of bone screws retained on the holder by two retainers.

The particular examples disclosed herein have been selected by the inventors simply to describe and illustrate examples of the invention and are not intended to limit the scope of the invention or its protection, which is to be given the full breadth of the appended claims and any and all equivalents thereof. Artisans of ordinary skill will appreciate that various modifications and alternatives for the described and illustrated examples can be developed in light of the overall teachings of the disclosure. These modifications and alternatives are within the scope of the invention. For example, an element or elements of one example holder or kit described and illustrated herein can be combined with an element or elements of another example holder or kit without departing from the scope of the invention. Similarly, a step or steps of one example method described and illustrated can be combined with a step or steps of another example without departing from the scope of the invention.

1. A holder for a bone screw, comprising:
a main body having a first surface and a second surface, the main body defining a retainer having first and second arms, each of the first and second arms comprising a flap of the main body that is movable from a first configuration, in which the respective arm is coplanar with the adjacent portions of the main body, and a second configuration in which the respective arm extends away from the adjacent portions of the main body.

2. The holder of claim 1, wherein the first arm defines a first terminal surface and the second arm defines a second terminal surface; and
wherein the first and second terminal surfaces cooperatively define a channel extending between the first and second arms when each of the first and second arms are in the respective second configuration.

3. The holder of claim 2, wherein the first and second terminal surfaces contact each other when each of the first and second arms are in the respective first configuration.

4. The holder of claim 1, wherein the first arm defines a first arcuate edge.

5. The holder of claim 4, wherein the second arm defines a second arcuate edge.

6. The holder of claim 5, wherein the first and second arms cooperatively define a stilted arch when each of the first and second arms are in the respective first configuration.

7. The holder of claim 1, wherein main body defines a recess when each of the first and second arms are in the respective second configuration.

8. The holder of claim 7, wherein the recess extends through the entire thickness of the main body.

9. The holder of claim 7, wherein the recess has an arcuate shape.

10. The holder of claim 9, wherein the main body defines a projection that extends into the recess.

11. The holder of claim 1, wherein the main body defines a second retainer having third and fourth arms, each of the third and fourth arms comprising a flap of the main body that is movable from a first configuration, in which the respective arm is coplanar with the adjacent portions of the main body, and a second configuration in which the respective arm extends away from the adjacent portions of the main body.

12. The holder of claim 1, wherein the main body comprises a flexible material.

13. The holder of claim 12, wherein the main body comprises plastic.

14. The holder of claim 12, wherein the main body comprises high density polyethylene.

15. A holder for a bone screw, comprising:
a main body having a first surface and a second surface, the main body defining a first retainer having a first arm and a second arm and a second retainer having a third arm and a fourth arm;
each of the first, second, third, and fourth arms comprising a flap of the main body that is movable from a first configuration in which the respective arm is coplanar with the adjacent portions of the main body and a second configuration in which the respective arm extends away from the adjacent portions of the main body;
a first terminal surface defined by the first arm and a second terminal surface defined by the second arm cooperatively defining a first channel extending between the first and second arms when each of the first and second arms are in the respective second configuration;
a third terminal surface defined by the third arm and a fourth terminal surface defined by the fourth arm cooperatively defining a second channel extending between the third and fourth arms when each of the third and fourth arms are in the respective second configuration.

16. The holder of claim 15, wherein the first and second arms extend away from the main body in a first direction when each of the first and second arms are in the respective second configuration.

17. The holder of claim 16, wherein the third and fourth arms extend away from the main body in a second direction when each of the third and fourth arms are in the respective second configuration.

18. The holder of claim 17, wherein the second direction is the same as the first direction.

19. The holder of claim 17, wherein the second direction is different from the first direction.

20. A holder for a bone screw, comprising:
a main body having a first surface and a second surface, the main body defining at least one single retainer clip and at least one double retainer clip;
each single retainer clip comprising a retainer having a first arm and a second arm movable between a first configuration in which the respective arm is coplanar with the adjacent portions of the main body and a second configuration in which the respective arm extends away from the adjacent portions of the main body;
each double retainer clip comprising a first retainer having a third arm and a fourth arm and a second retainer having a fifth arm and a sixth arm, each of the third, fourth, fifth, and sixth arms movable between a first configuration in which the respective arm is coplanar with the adjacent portions of the main body and a second configuration in which the respective arm extends away from the adjacent portions of the main body;
each arm of each of the single retainer clips and each arm of each of the double retainer clips biased toward returning to the first configuration in which the respective arm is coplanar with adjacent portions of the main body.
